(19) United States
(12) Patent Application Publication
(10) Pub. No.: US 2007/0154990 A1
(43) Pub. Date: Jul. 5, 2007

(54) Assays for identifying inhibitors of fatty acid amide hydrolase

(76) Inventors: Yuren Wang, Plainsboro, NJ (US); Philip G. Jones, Danvers, MA (US); Jun Xu, Monmouth Junction, NJ (US); Albert J. Uveges, Hatboro, PA (US)

Correspondence Address: KIRKPATRICK & LOCKHART PRESTON GATES ELLIS LLP, STATE STREET FINANCIAL CENTER, ONE LINCOLN STREET, BOSTON, MA, 02111-2950, US

(21) Appl. No.: 11/635,806

(22) Filed: Dec. 7, 2006

Related U.S. Application Data
(60) Provisional application No. 60/748,431, filed on Dec. 7, 2005.

(51) Int. Cl. C12P 21/06

(52) U.S. Cl. 435/69.1

(57) ABSTRACT

Assays for screening compounds suspected of binding fatty acid amide hydrolase (FAAH) using scintillation proximity assay (SPA) technology are described. The assays utilize the interactions of reversible inhibitors such as [³H]—R(+)-methanandamide (MAEA) and membrane-associated FAAH, such as FAAH-containing microsomes, to evaluate the displacement activity of candidate FAAH inhibitors. The assays are specific for FAAH where FAAH binding compounds can be detected. Various embodiments of the assay have been validated and demonstrated to be simple, sensitive and amenable to high-throughput screening. Kits for performing the assays are also disclosed.

This application claims the benefit of the filing date of U.S. Provisional Patent Application Ser. No. 60/748,431, filed on Dec. 7, 2005, the entire disclosure of which is incorporated by reference herein for all purposes.

### FIELD

The present teachings relate to assay methods for screening for inhibitors of fatty acid amide hydrolase (FAAH). In particular, the present teachings relate to binding assays for human FAAH using a variation of scintillation proximity assay (SPA) technology.

### BACKGROUND

Fatty acid amide hydrolase (FAAH) is a membrane-associated member of the serine hydrolase superfamily. It is an integral membrane protein with an N-terminal transmembrane domain and two hydrophobic motifs which may be responsible for its membrane anchorage (See Bracey, M. H., et al., Structural adaptations in a membrane enzyme that terminates endocannabinoid signaling, Science, 2002, 298(5599): 1793-6). Evidence from cellular fractionation and functional studies have demonstrated that FAAH is predominantly localized to the intracellular microsomal fractions of cells (See Ueda, N., et al., Partial Purification and Characterization of the Porcine Brain Enzyme Hydrolyzing and Synthesizing Anandamide, J. Biol. Chem., 1995, 270(40): 23823-23827 and Ramarao, M. K., et al., A Fluorescence-based Assay for Fatty Acid Amide Hydrolase Compatible with High-throughput Screening, Analytical Biochemistry, 2005, in press).

The substrates for FAAH include several physiologically important endogenous fatty acid amides, for example, anandamide (arachidonyl ethanolamide, AEA), N-palmitoylethanolamine (PEA) and N-oleoylethanolamine (OEA) (See Deutsch, D. G., N. Ueda, and S. Yamamoto, The fatty acid amide hydrolase (FAAH), [erratum appears in Prostaglandins Leukot Essent Fatty Acids, 2003 Jan., 68(1):69] Prostaglandins Leukotrienes & Essential Fatty Acids, 2002, 66(2-3): 201-10; McKinney, M. K. and B. F. Cravatt, Structure and Function of Fatty Acid Amide Hydrolase, Annual Review of Biochemistry, 2005, 74: 411-32 and Bisogno, T., L. De Petrocellis, and V. Di Marzo, Fatty acid amide hydrolase, an enzyme with many bioactive substrates. Possible therapeutic implications, Current Pharmaceutical Design, 2002, 8(7): 533-47). These endogenous fatty acid amides have anti-inflammatory activities, and are involved in a variety of physiological activities such as pain sensation, mood modulation, sleep-induction, and appetite-suppression (See Devane, W., et al., Isolation and structure of a brain constitute that binds to the cannabinoid receptor, Science, 1992, 258: 1946-1949; Cravatt, B. F., et al., Chemical characterization of a family of brain lipids that induce sleep, Science, 1995, 268: 1506-1509; Kuehl, F. A., et al., The identification of N-(2-hydro-xyethyl)-palmitamide as a naturally occurring anti-inflammatory agent, J Am Chem Soc, 1957, 79: 5577-5578 and Rodriguez de Fonseca, F., et al., An anorexic lipid mediator regulated by feeding, Nature, 2001, 414(6860): 209-212).

Several lines of evidence have demonstrated that FAAH plays a key role in regulating the physiological tones and activities of these endogenous fatty acid amides in vivo. Thus, FAAH has been regarded as an attractive therapeutic target for the treatment of pain, anxiety, inflammation and other disorders (See Clement, A. B., et al., Increased seizure susceptibility and proconvulsant activity of anandamide in mice lacking fatty acid amide hydrolase, Journal of Neuroscience, 2003, 23(9): 3916-23; Cravatt, B. F., et al., Functional disassociation of the central and peripheral fatty acid amide signaling systems, Proceedings of the National Academy of Sciences of the United States of America, 2004, 101(29): 10821-6; Cravatt, B. F., et al., Supersensitivity to anandamide and enhanced endogenous cannabinoid signaling in mice lacking fatty acid amide hydrolase, Proceedings of the National Academy of Sciences of the United States of America, 2001, 98(16): 9371-6; Lichtman, A. H., et al., Pharmacological activity of fatty acid amides is regulated, but not mediated, by fatty acid amide hydrolase in vivo, Journal of Pharmacology & Experimental Therapeutics, 2002, 302(1): 73-9; Huitron-Resendiz, S., et al., Characterization of the sleep-wake patterns in mice lacking fatty acid amide hydrolase, Sleep, 2004, 27(5): 857-65 and Kathuria, S., et al., Modulation of anxiety through blockade of anandamide hydrolysis, Nature medicine, 2003, 9(1): 76-81).

Efforts are underway to discover FAAH inhibitors with drug-like properties. The crystal structure of FAAH reveals an unusual serine-serine-lysine catalytic triad, and a long substrate binding channel that can fully accommodate an arachidonyl moiety (See McKinney, M. K. and B. F. Cravatt, Structure and Function of Fatty Acid Amide Hydrolase, Annual Review of Biochemistry, 2005, 74: 411-32 and Bracey, M. H., et al., Structural adaptations in a membrane enzyme that terminates endocannabinoid signaling, Science, 2002, 298(5599): 1793-6). FAAH inhibitors are likely either to bind to the enzyme acyl chain pocket, or to target the active site via the cytoplasmic channel, to obtain the necessary selectivity for FAAH over other lipid hydrolases (See Bracey, M. H., et al., Structural adaptations in a membrane enzyme that terminates endocannabinoid signaling, Science, 2002, 298(5599): 1793-6).

Most of the potent FAAH inhibitors described thus far are electrophiles that irreversibly inhibit FAAH by covalently linking to the catalytic nucleophile, Ser-241, at the active site (See Kathuria, S., et al., Modulation of anxiety through blockade of anandamide hydrolysis, Nature medicine, 2003, 9(1): 76-81; Patricelli, M. P., M. A. Lovato, and B. F. Cravatt, Chemical and mutagenic investigations of fatty acid amide hydrolase: evidence for a family of serine hydrolases with distinct catalytic properties, Biochemistry, 1999, 38(31): 9804-12; Ueda, N., et al., Partial Purification and Characterization of the Porcine Brain Enzyme Hydrolyzing and Synthesizing Anandamide, J. Biol. Chem., 1995, 270(40): 23823-23827; Boger, D. L., et al., Trifluoromethyl ketone inhibitors of fatty acid amide hydrolase: a probe of structural and conformational features contributing to inhibition, Bioorganic & Medicinal Chemistry Letters, 1999, 9(2): 265-70 and Boger, D. L., et al., Exceptionally potent inhibitors of fatty acid amide hydrolase: the enzyme responsible for degradation of endogenous oleamide and anandamide, Proceedings of the National Academy of Sciences of the United States of America, 2000, 97(10): 5044-9). However, such irreversible inhibitors have not been pursued for drug development. More recently, several potent and selective classes of reversible FAAH inhibitors have been identified and found to be efficacious in animal models of pain (See Boger, D. L., et al., Discovery of a Potent, Selective, and Efficacious Class of Reversible-Ketoheterocycle Inhibitors of Fatty Acid Amide Hydrolase Effective as Analgesics, J. Med. Chem., 2005, 48: 1849-1856).

Current efforts to develop assays to identify FAAH inhibitors are predominantly focused on functional assays that monitor FAAH activity using either radiolabeled or fluorogenic substrates (See Ueda, N., et al., Partial Purification and Characterization of the Porcine Brain Enzyme Hydrolyzing and Synthesizing Anandamide, J. Biol. Chem., 1995, 270(40): 23823-23827; Deutsch, D. G. and S. A. Chin, Enzymatic synthesis and degradation of anandamide, a cannabinoid receptor agonist, Biochemical Pharmacology, 1993, 46(5): 791-796; Koutek, B., et al., Inhibitors of arachidonoyl ethanolamide hydrolysis, J. Biol. Chem., 1994, 269(37): 22937-22940; Lang, W., et al., High-Performance Liquid Chromatographic Determination of Anandamide Amidase Activity in Rat Brain Microsomes, Analytical Biochemistry, 1996, 238(1): 40-45; Omeir, R. L., et al., Arachidonoyl ethanolamide-[1,2-14C] as a substrate for anandamide amidase, Life Sciences, 1995, 56(23-24): 1999-2005; Maccarrone, M., M. Bari, and A. F. Agro, A sensitive and specific radiochromatographic assay of fatty acid amide hydrolase activity, Analytical Biochemistry, 1999, 267(2): 314-8; Boldrup, L., et al., A simple stopped assay for fatty acid amide hydrolase avoiding the use of a chloroform extraction phase, Journal of Biochemical & Biophysical Methods, 2004, 60(2): 171-7; and Ramarao, M. K., et al., A Fluorescence-based Assay for Fatty Acid Amide Hydrolase Compatible with High-throughput Screening, Analytical Biochemistry, 2005, in press). To date, there are no binding assays reported for FAAH, even though direct evidence of the binding of an inhibitory compound to its target is essential for understanding the structure-activity relationships of enzyme inhibitors, as well as their mechanism(s). There is thus a need in the art for binding assays to screen for and detect inhibitors of FAAH activity, especially human FAAH activity.

### SUMMARY

One aspect of the present teachings features a method for identifying a compound capable of binding to fatty acid amide hydrolase (FAAH). The method generally comprises the following steps: (1) incubating a membrane-associated FAAH with a labeled, known reversible inhibitor of FAAH activity in the presence of at least one type of scintillation proximity assay (SPA) bead and in the presence of a test compound suspected of being capable of binding to the FAAH, wherein when the labeled known reversible inhibitor is bound to the FAAH, substantially no signal is generated from the SPA beads and wherein the presence of unbound labeled known reversible inhibitor in the assay medium causes the scintillation proximity beads to generate a detectable signal; (2) measuring a signal from the SPA beads; (3) comparing the signal so measured to a signal measured in a control assay incubated in the absence of the test compound; and (4) determining if the test compound binds to the FAAH by correlating the signals, for example, an increase in signal compared to the control assay indicates an increase in binding of the test compound.

In certain embodiments, the membrane-associated FAAH comprises a lipid bilayer system, for instance, a liposome, a microsome, a cell ghost, a cell membrane, or a cell membrane fraction. The SPA bead(s) can include at least one of yttrium silicate, polyvinyltoluene, wheat germ agglutinin, polylysine, and polyethyleneimine. The reversible inhibitor can be, for example, methanandamide, URB-597, CAY10400, CAY10402, or CAY-10435. In particular embodiments, the reversible inhibitor is a substrate analog that is not substantially hydrolyzed by the FAAH enzyme. The test compound can bind to the active site of FAAH in some embodiments.

In certain embodiments, the label is a radioactive label comprising at least one of ¹⁴C and ³H. The detectable signal can comprise an emission of light, which can include at least one visible wavelength. In particular embodiments, the signal can be detected in a high throughput system.

Another aspect of the present teachings features a kit for assaying a compound for its ability to bind to a FAAH enzyme. The kit can include, for example, a membrane-associated FAAH; at least one type of scintillation proximity assay (SPA) beads; a labeled, known reversible inhibitor of FAAH; and instructions for a scintillation proximity assay-based method for detecting a compound that binds to the FAAH. In various embodiments, the kit can further include reaction vessels and/or a detector for detecting a signal generated by the scintillation proximity beads, and optionally, a computer system for storing and analyzing results obtained from the assaying. The kit can also include at least one known inhibitor as a standard. In specific embodiments, the labeled inhibitor included in the kit is [3H]—R(+)-methanandamide, and the SPA beads included in the kit comprise one or more of yttrium silicate, polyvinyltoluene, wheat germ agglutinin, polylysine, and polyethyleneimine.

Another type of kit for detecting a compound capable of binding to a FAAH enzyme can include a vector comprising a FAAH gene for expressing in a cell; at least one scintillation proximity assay bead; a labeled, known competitive inhibitor of FAAH; and instructions for a scintillation proximity assay-based method for detecting a compound that binds to the FAAH. In specific embodiments, the vector comprises pHTOP-FAAH-His6 for expression in a mammalian cell.

Other features and advantages of the present teachings will be more fully understood by reference to the following non-limiting drawings, detailed description, examples, and claims.

### BRIEF DESCRIPTION OF THE DRAWINGS

A skilled artisan will understand that the drawings described below are for illustration purposes only and are not necessarily to scale unless otherwise understood. The drawings are not intended to limit the scope of the present teachings in any way.

FIG. 1. Schematic representation of an exemplary scintillation proximity assay (SPA) for FAAH. This embodiment of the SPA assay includes three components: FAAH-containing microsomes, FAAH ligand such as [³H]-MAEA, and WGA SPA beads. When the three are incubated together, [³H]-MAEA binds predominately to the FAAH-containing microsomes. However, in the presence of a FAAH inhibitor, the [³H]-MAEA is displaced from the enzyme's active site. The released [³H]-MAEA will interact non-specifically with WGA SPA beads to stimulate a SPA signal.

FIG. 2. Results of kinetic analysis of anandamide hydrolysis by FAAH-containing microsomes:

Panel (A) Dose-dependent inhibition of FAAH microsome catalyzed AEA hydrolysis by MAEA. 0.5 μg of human FAAH microsomes were incubated with 1 μM [³H]-AEA substrate and indicated concentrations of MAEA in assay buffer at room temperature for 2 hours. Released [³H]-ethanolamine was extracted by chloroform/methanol and counted.

Panel (B) Effect of MAEA on the kinetics of AEA hydrolysis by FAAH-containing microsomes. 0.5 μg of the microsomes comprising FAAH were incubated with the indicated [³H]-AEA concentrations for 30 minutes with or without 10 μM of MAEA. Released [³H]-ethanolamine was measured in a scintillation counter.

Panel (C) Lineweaver Burk Plot of AEA hydrolysis by FAAH-containing microsomes in the presence of 10 μM of MAEA. Each data point represents mean±SD (n=3).

FIG. 3. One embodiment of a FAAH SPA assay. The reactions were carried out by incubating WGA beads (0.5 mg), [³H]-MAEA (100 nM), or FAAH-containing microsomes (5 μg), or URB-597 (1 μM) in assay buffer consisting of 50 mM HEPES, 1 mM EDTA, pH 7.4, 0.1% BSA in a total volume of 200 μL for 3 hours at room temperature. The radioactivity was counted on a TopCount™ for one minute per sample. Each point shown is the mean±SD of triplicates from at least two independent experiments.

FIG. 4. Effect of microsomal preparations prepared from cells expressing FAAH, inactive S241A FAAH mutant, or no FAAH (vector control) on the binding of [³H]-MAEA to WGA SPA beads. The relative activity of FAAH-containing microsomes or those prepared from cells expressing FAAH-S241A or vector control was measured by increasing the microsomal protein concentration ranging from 0-10 μg/assay. 100 nM of [³H]-MAEA and 0.5 mg of WGA SPA beads were incubated with indicated concentrations of microsomes at 25° C. for 3 hours. Radioactivity was measured in a TopCount™. The SPA signal (CPM) was plotted against amount of microsomal protein in the assay. Each point is the mean±SD of triplicate data from at least two independent experiments.

FIG. 5. Selection of SPA beads for FAAH SPA.

Panel (A) Binding of [³H]-MAEA to the different SPA beads. Assays were performed by incubating 5 μg of FAAH-containing microsomes with 100 nM [³H]-MAEA and 0-2 mg SPA beads in assay buffer at room temperature for 3 hours.

Panel (B) Effects of SPA beads in the SPA assay. Standard SPA assays were conducted as described in materials and methods, 0.5 mg SPA beads were incubated in the absence or presence of 5 μg of FAAH-containing microsomes. Each point represents the mean±SD (n=3).

FIG. 6. Effects of [³H]-MAEA concentration on SPA signal. Assays performed in the presence or absence of 2 μg of FAAH-containing microsomes are shown. Reactions were performed with the indicated [³H]-MAEA concentration as described in Materials and Methods. Specific binding was determined by subtracting the counts of FAAH microsomes from the total counts without FAAH microsomes (buffer).

FIG. 7. Inhibition of the binding of [³H]-MAEA to FAAH-containing microsomes by FAAH inhibitors in SPA. The assay was carried out by incubating 5 μg of FAAH microsomes with 100 nM [³H]-MAEA and 0.5 mg of WGA beads in 50 mM HEPES, 1 mM EDTA, pH 7.4, 0.1% BSA, with inhibitors in a 200 μL total reaction volume, at room temperature for 3 hours. The final concentration of DMSO in the assay was 0.5%. IC₅₀ values were calculated by fitting the curve to a non-linear regression equation. Each point is the mean±SD of triplicate data from at least two independent experiments.

### DETAILED DESCRIPTION OF ILLUSTRATIVE EMBODIMENTS

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

The following abbreviations may be used herein: FAAH, fatty acid amide hydrolase; SPA, scintillation proximity assay; AEA, arachidonyl ethanolamide, anandamide; MAEA, R-(+)-methanandamide; WGA, wheat germ agglutinin; HTS, high-throughput screening; CHO, Chinese hamster ovary; BSA, bovine serum albumin; CPM, counts per minute; PVT, polyvinyltoluene; PEI, polyethyleneimine; and YSi, yttrium silicate.

The present teachings feature assays and kits for screening and/or detecting compounds that inhibit FAAH activity. Three components are employed in the assay: a membrane-associated FAAH, a SPA bead, and a labeled, known inhibitor of FAAH that reversibly binds to FAAH for example, a radiolabeled, known inhibitor of FAAH. In various embodiments, the latter component exhibits a strong and specific binding affinity for FAAH and exhibits a nonspecific interaction with the SPA beads upon release from FAAH into the assay medium.

When the three components are incubated together, a radiolabeled known reversible inhibitor of FAAH binds predominantly to the membrane-associated FAAH. Because only a small portion of free radiolabel is available to bind to the beads, low SPA signals are observed under these conditions. However, in the presence of FAAH inhibitors, which may compete with known reversible inhibitor for binding to the FAAH, the radiolabeled known reversible inhibitor is displaced and released into the assay solution where it interacts with the SPA beads, thus producing higher SPA signals. Therefore, unlike traditional binding assays that detect the radiolabel remaining bound to a target, the present teachings can provide an assay that detects an increase in the free radiolabel concentration that occurs in the presence of an inhibitor.

Furthermore, the SPA assay of the present teachings is different from classical SPA binding assays in other aspects. First, the membrane bound FAAH does not bind to the SPA beads, whereas in the classical SPA, the target protein is tethered to the SPA beads by physical affinity or chemical linkages. For example, streptavidin-coated beads capture biotin, antibody-coated beads capture the cognate antigen, and WGA-coated beads immobilize glycosylated proteins such as G protein-coupled receptors. Second, the present FAAH assay can exploit the non-specific interaction that certain reversible FAAH inhibitors have for the SPA beads. In contrast, the classical assays require that the radiolabels should have an extremely low binding affinity for the beads to reduce the background. Consequently, in the present assays, the selected reversible FAAH inhibitors are expected to increase the SPA signals upon release into the assay medium, whereas in the classical SPA binding assay, the displacement of agonists or antagonists results in a decreased SPA signal.

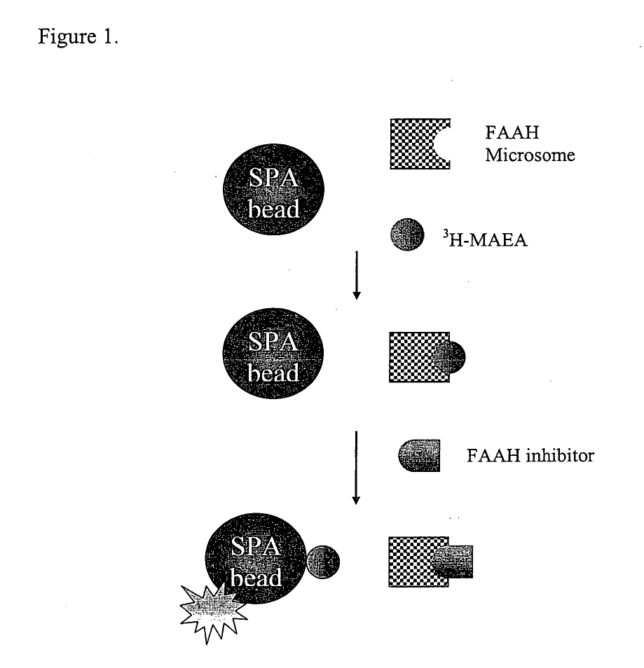

In an exemplary embodiment shown schematically in FIG. 1 and described in detail in the examples, it has been shown that the competitive, non-hydrolyzed FAAH inhibitor, R(+)-methanandamide (MAEA), binds specifically to FAAH-containing microsomes. Co-incubation with a FAAH inhibitor, for example URB-597, competitively displaced the MAEA on the FAAH-containing microsomes. Accordingly, an exemplary embodiment of the present teachings features assays based on a Scintillation Proximity Assay (SPA), that utilizes the specific interactions of [³H]—R(+)-methanandamide (MAEA) and FAAH-containing microsomes to evaluate the displacement activity of putative FAAH inhibitors. Released radiolabel can be readily detected through interactions with the SPA assay beads. The assays are specific for FAAH, as shown by the observations that microsomes prepared from cells expressing only inactive FAAH had no significant ability to bind [³H]-MAEA, nor did microsomes from cells not expressing FAAH. In addition, the binding of [³H]-MAEA to FAAH-containing microsomes can be abolished by selective FAAH inhibitors in a dose-dependent manner. The assays have been validated and demonstrated to be simple, sensitive and amenable to high-throughput screening.

Thus, one aspect of the present teachings provides methods for identifying a compound capable of binding to fatty acid amide hydrolase (FAAH), the method comprising: (1) providing a membrane-associated FAAH enzyme; (2) providing at least one type of SPA beads; (3) incubating the membrane-associated FAAH enzyme with a labeled, known reversible inhibitor of FAAH activity in the presence of the SPA beads and in the presence of a test compound, which can be suspected of being capable of binding to the FAAH, and thereby displacing the labeled, known reversible FAAH inhibitor, wherein the displaced labeled known reversible inhibitor causes the SPA beads to generate a detectable signal when in sufficiently close proximity to the beads; (3) measuring a signal from the SPA beads; and (4) comparing the signal measured in the presence of the test compound with a signal measured in a control assay incubated in the absence of the test compound. More specifically, the control assay can comprise (1) providing a membrane-associated FAAH enzyme; (2) providing at least one type of SPA beads; (3) incubating the membrane-associated FAAH enzyme with a labeled, known reversible inhibitor of FAAH activity in the presence of the SPA beads and in the absence of the test compound. An increase in signal in the presence of the test compound indicates that the test compound has bound to the membrane-associated FAAH and displaced the known, reversible FAAH inhibitor.

In some embodiments, the FAAH enzyme exists in a state similar to that found in nature, i.e., it is membrane-associated. Methods of studying membrane-associated proteins, and enzymes in particular, are known in the art. In certain embodiments, the FAAH is provided in the form of microsomal preparations. In various embodiments, the enzyme can be associated with lipsomes of various types as are known in the art, or with cellular membrane fractions or preparations. Whole cell membrane and ghost cells can be used in connection with the methods provided herein. Other means for providing a membrane-associated enzyme are known in the art and suitable for use in the present teachings. In particular embodiments, purified FAAH enzyme can be utilized.

The FAAH preparations or fractions suitable for use in the present teachings are membrane fractions or preparations, for example, microsomal preparations from cells known to produce the FAAH activity. Included herein are methods that can involve expression of FAAH-encoding nucleic acids provided in a vector, such as pHTOP-FAAH-His6. Also useful herein for certain embodiments and for generating variations of those embodiments not specifically exemplified, but consistent with the teachings herein, are vectors such as pHTOP-FAAH-S241A-His6 plasmid. This vector is known in the art and results in the expression of an inactive FAAH. Microsomal or membrane preparations from cells harboring this vector can be useful in distinguishing specific and nonspecific binding aspects of assays as exemplified herein. The skilled artisan will appreciate how to use such vectors in practicing the assays disclosed herein, as well as how to use the vector controls.

Methods of preparing the microsomes and other membrane preparations are available to those of skill in the art, as are methods for establishing cell lines that stably express the desired construct or constructs.

Scintillation proximity assay (SPA) is art-known methodology (see, e.g., U.S. Pat. No. 4,568,649) that utilizes scintillation beads, or microspheres containing scintillant, which emit light, preferably in the visible spectrum, for example, in the blue region of the visible spectrum. SPA beads are particularly well-adapted for use with photomultiplier tube-based scintillation counters. Commercially available instruments include, for example, the TopCount™ or MicroBeta™ instruments, which are adapted for rapid reading of multiple samples. Accordingly, SPA-based methodologies can be well-suited for high-throughput screening.

Various types of SPA beads are commercially available—two common types comprise, respectively, yttrium silicate (YSi) and polyvinyltoluene (PVT). The surfaces of the beads can be modified to facilitate different binding affinities. For example, each bead type can be adapted through the use of certain biological molecules to make it better suited for certain assays, such as receptor binding, kinase assay, various specific molecular interactions (e.g. biotin and streptavidin or other biotin binding protein), or particular radioimmunoassays. As has been shown, a variety of beads types are compatible for use with the methods of the present teachings. In various embodiments, the assays use either YSi beads or PVT beads. In certain embodiments, the assays use beads whose surfaces are modified with wheat germ agglutinin, polyethyleneimine or polylysine. In particular embodiments, PVT beads with wheat germ agglutinin, or PVT beads with wheat germ agglutinin and polyethyleneimine are used. Such beads are commercially available from Amersham Bioscience as PVT-WGA and PVT+PEI-WGA type B, respectively.

The methods described herein involve incubating the FAAH preparation with an inhibitor and optionally, a test compound depending on whether the assay is the control assay. Incubation for such assays can be readily modified or optimized by the skilled artisan. Typically, a three hour incubation is performed to allow ample time for all components to reach steady state with respect to their binding.

The inhibitors for use with the current present teachings can be of several types. Labeled known inhibitors can be used to initially bind to the FAAH, preferably but not necessarily at its active site, in conjunction with the initial steps of the assays provided. Unknown inhibitors (also referred to herein alternatively as putative inhibitors, test compounds, candidate compounds, compounds of interest, and the like) are used in the instant methods as the objects of the screening method.

With respect to the known inhibitors used in the methods, suitable inhibitors include reversible inhibitors or inhibitors that can be displaced from the FAAH enzyme by a compound of interest that binds to the FAAH enzyme, particularly at the active site thereof. Known inhibitors should be substantially stable in the presence of the FAAH activity and not substantially degraded or hydrolyzed thereby. In some embodiments, the known inhibitor is less than 10% hydrolyzed after exposure to active FAAH for a time period at least equal to the length of the assay. In various embodiments, the known inhibitor is less than about 5% hydrolyzed, less than about 4% hydrolyzed, less than about 3% hydrolyzed, or less than about 2% hydrolyzed after exposure to active FAAH. In particular embodiments, inhibitors can remain substantially undegraded or nonhydrolyzed after prolonged exposure to the FAAH activity, for example, up to 5 hours or longer.

In certain embodiments, the known inhibitor is an analog or derivative of a natural or synthetic substrate of the FAAH. The analog or derivative can be resistant to hydrolysis by the FAAH. For example, analogs of anandamide are used as inhibitors in some embodiments.

The known inhibitors can be reversible competitive inhibitors. In some embodiments, the inhibitor alters the kinetic relationship between the FAAH enzyme and its substrate. In certain embodiments, the known inhibitor alters the apparent Michaelis constant (Kₘ, the substrate concentration at which the velocity of the enzyme reaction is one half maximal) for the substrate but does not substantially alter the maximal velocity of the enzyme-catalyzed reaction (Vₘₐₓ). Thus, the Kₘ is increased by the known inhibitor, but the Vₘₐₓ is not substantially altered.

Suitable reversible competitive inhibitors include, but are not limited to, methanandamide, URB-597, CAY10400, CAY 10402, and CAY-10435. In particular embodiments, labeled methanandamide, for example, [³H]-methanandamide, is used. In certain embodiments, the known inhibitor is [³H]—R(+)-methanandamide, which can include a specific activity such as 60 Ci/mmol.

The compounds of interest that can be detected by the methods of the present teachings have the ability to displace a known inhibitor that is bound to the FAAH. Such compounds can be competitive, non-competitive or uncompetitive FAAH inhibitors. The displacement can happen rapidly, which allows for shorter assay times. In various embodiments, the assays are conducted in about 3 hours or less, having reached a steady state with respect to inhibitor displacement. In certain embodiments, where the displacement of bound inhibitor has reached a steady state within about 2 hours or less, a test compound can be accurately assayed at that time. Some assays will allow accurate measurements to be taken after less than about 2 hours, less than about 90 minutes, or less than about 60 minutes. Certain assays can allow an accurate measurement to be taken after less than 60 minutes—such as less than about 30 minutes, less than about 20 minutes, or less than about 10 minutes. In various embodiments the unknown inhibitors are not subject to hydrolysis by the FAAH, but are as stable as, or more so than, the known inhibitor used in the assay method. The unknown inhibitors detected by the assay also can be metabolically stable, i.e., stable in vivo for sufficient time to allow a biological effect.

In some embodiments of the method, membrane-associated FAAH, for example, a microsomal preparation from cells expressing a FAAH, are contacted with a labeled inhibitor in the presence of SPA beads comprising a wheat germ agglutinin modified surface. The results are reproducible and can be suited for high-throughput screening of candidate compounds. The Z′ factor, which measures the reproducibility and HTS compatability of the assay, can be at least about 0.7 or greater, or about 0.73 or greater, or within a range of about 0.75-0.8, or about 0.8 or greater.

In another aspect, the present teachings provide a kit for assaying a compound for its ability to bind to a FAAH enzyme. The kit generally comprises: a membrane-associated FAAH; at least one scintillation proximity assay bead; a labeled, known reversible inhibitor of FAAH; and instructions for a scintillation proximity assay-based method for detecting a compound that binds to the FAAH. The FAAH enzyme preparation can comprise a membrane, for example, a microsomal preparation. A kit as described herein can be used to practice the methods of the present teachings.

Further, the components of the kit can be as described herein. For example, the competitive inhibitor of FAAH can be an analog or derivative of a natural or synthetic substrate of the FAAH enzyme, for example, methanandamide, URB-597, CAY 10400, CAY 10402 or CAY-10435. In particular embodiments, the labeled inhibitor is [3H]—R(+)-methanandamide.

In some embodiments, at least one scintillation proximity assay bead comprises at least one of yttrium silicate, polyvinyltoluene, wheat germ agglutinin, polylysine, and polyethyleneimine. In certain embodiments, the SPA bead comprises polyvinyl toluene and wheat germ agglutinin. Multiple SPA beads can, of course, be used, and more complex assays can be practiced where more than one type of SPA bead is used in a single assay. Because the methods are particularly adaptable to HTS methods, in certain embodiments the methods are those wherein the Z′ factor is calculated to be greater than about 0.7, or about 0.8 or greater.

In various embodiments, the kits comprise reaction vessels and/or a detector for detecting a signal generated by the scintillation proximity assay bead(s), and optionally, a computer system for storing and analyzing results obtained from the assay. Reaction vessels can be convenient and easy to manipulate either manually or in automated systems such as high-throughput screening systems. In some embodiments, the reaction vessels comprise the wells of a multiwell plate, for example, each well of a 96-well microplate. The use of such multiwell vessels is known to those of skill.

The kit can also include at least one known inhibitor as a standard. Multiple known inhibitors can be useful to gain a better comparison of the binding properties, for example, binding affinity, of the putative inhibitor or test compound relative to known inhibitors under actual assay conditions. In certain embodiments, the kit includes [³H]—R(+)-methanandamide as a labeled inhibitor. The kits can include scintillation proximity assay beads comprising at least one of yttrium silicate (YSi), polyvinyltoluene (PVT), wheat germ agglutinin, polylysine and polyethyleneimine. In particular embodiments, the kits include PVT beads with wheat germ agglutinin attached. Kits optionally include standard reagents for practicing the methods such as buffer components, DMSO, and BSA additions.

In various embodiments, the kits permit implementation of an assay method of the present teachings through the establishment of a cell line expressing the FAAH. For example, such a kit can comprise a vector comprising a FAAH gene for expressing in a cell; at least one scintillation proximity assay bead; a labeled, known competitive inhibitor of FAAH; and instructions for a scintillation proximity assay-based method for detecting a compound that binds to the FAAH. In utilizing such kits, it should be understood that the skilled artisan would use the vector to establish a cell line, for example, a stable cell line expressing the FAAH enzyme. A FAAH preparation, such as a membrane-associated FAAH, then can be prepared by the practitioner according to standard methods. The FAAH preparation then can be used with the remaining components of the kit to practice the binding assays described herein. In certain embodiments, the kits provide vectors such as the pHTOP-FAAH-His6 which comprise a FAAH encoding sequence for expression in a mammalian cell.

The following examples are offered for illustrative purposes and are not intended to limit the present teachings in any manner. Those of skill in the art will readily recognize a variety of noncritical parameters which can be changed or modified to yield essentially the same results.

### EXAMPLE 1

### Exemplary Assay Assembly

Materials

[³H]—R(+)-methanandamide (60 Ci/mmol) was obtained from American Radiolabeled Chemicals Inc. (St. Louis, Mo.). SPA beads were obtained from Amersham Biosciences (Piscataway, N.J.). White OptiPlate™-96 polystyrene microplates were obtained from PerkinElmer Life Science (Boston, Mass.). FAAH inhibitors URB-597, CAY-10400, CAY-10402 and CAY-10435 were obtained from Cayman Chemicals (Ann Arbor, Mich.). Arachidonoyl trifluoromethyl ketone (ATFK) and other chemicals were obtained from Sigma-Aldrich (St Louis, Mo.).

Expression Constructs and Establishment of Stable Cell Lines

The construction of pHTOP-FAAH-His6 and pHTOP-FAAH S241A-His6 plasmids for mammalian expression has been described previously (See Ramarao, M. K., et al., A Fluorescence-based Assay for Fatty Acid Amide Hydrolase Compatible with High-throughput Screening, Analytical Biochemistry, 2005, in press). Briefly, CHO cells were transfected with linearized pHTOP-FAAH and pHTOP-FAAH-S241A DNA using Lipofectamine 2000 reagent according to the manufacturer's protocol (Invitrogen). The cells were selected at various concentrations of methotrexate for two-three weeks. Colonies were then isolated and cultured in MEM-α medium containing 10% dialyzed fetal calf serum, 100 IU/mL penicillin, 100 μg/mL streptomycin, 1 mg/mL G418, 2 mM L-glutamine, 15 mM HEPES, pH 7.4, and 100 nM methotrexate.

Preparation of Microsomal Fractions from CHO Cells

CHO cells expressing FAAH, the inactive FAAH-S241A, or empty vector were grown in suspension. Cells were collected by centrifugation at 2000 g for 10 minutes at 4° C. in a Sorvall high-speed centrifuge. The cell pellet was then washed 3 times with PBS and resuspended in buffer containing 50 mM HEPES, pH 7.4, 1 mM EDTA, 1 μM pepstatin, 100 μM leupeptin, 0.1 mg/mL aprotinin. The cells were sonicated 5 times for 10 seconds at 15-second intervals and the cell lysate was further centrifuged at 12000 g for 20 minutes at 4° C. The supernatant was centrifuged at 100,000 g for 30 minutes in a SW28 rotor in a Beckman Optima LE-80K centrifuge. The pellet obtained was the microsomal fraction, which was resuspended in ice-cold buffer. Protein concentration was measured by the BCA method (Pierce) and the microsomal suspension adjusted to 1 mg/mL and stored in aliquots at −80° C. until use.

SPA Assay

The SPA assay consisted of 100 nM [³H]-MAEA (60 Ci/mmol), 2.5 μg FAAH microsomes and 0.5 mg PVT-WGA beads in buffer (50 mM HEPES, pH 7.4, 1 mM EDTA and 0.1% BSA). The assays were performed in 96 well plates with a final volume of 200 μL. Components were added to the assay plates, which were agitated on a plate shaker for 3-4 hours at room temperature. The assays were then counted on a TopCount NXT (PerkinElmer, Boston, Mass.) for one minute per well. Results presented are the mean±S. E. of triplicate measurements from at least two independent experiments. The IC50 values were calculated using the GraphPad Prism 3.02 program, based on a sigmoidal dose-response equation. The screening window coefficient (Z′-factor) was calculated according to the following equation as previous described (See Zhang, J. H., T. D. Chung, and K. R. Oldenburg, A simple statistical parameter for use in evaluation and validation of high throughput screening assays, J Biomol Screen, 1999, 4: 67-73):
Z=1−(3σ_{c+}+3σ_{c-})/|μ_{c+}−μ_{c-}|
where, σ_{c+} and σ_{c-} are standard deviations of positive and negative controls and μ_{c+} and μ_{c-} are means of positive and negative controls respectively.

Assay of [³H]-anandamide Hydrolysis by FAAH Microsomes.

Microsomes (0.5 μg/assay) prepared from CHO cells expressing human FAAH, were incubated with the indicated concentrations of anandamide (ethanolamine 1-[³H]) for 30 min at 25° C. in 50 mM HEPES, pH 7.4, 1 mM EDTA and 0.1% BSA. The reaction was terminated by the addition of 2 volumes of chloroform/methanol (1:1, v/v). The samples were vortexed and centrifuged at 1000 g for 3 minutes. The aqueous phase, containing [³H]-ethanolamine, was collected and measured by liquid scintillation counting. Results are presented as the mean±S.E. of triplicate measurements from at least two independent experiments. The observed rate constants (apparent Kₘ) were calculated using GraphPad Prism 3.02, based on a Michaelis-Menten (one site binding hyperbola) equation Y=BₘₐₓX/[Kₘ+X].

### EXAMPLE 2

### R(+)-methanandamide is a Competitive FAAH Inhibitor and Binds to the FAAH Substrate Pocket

A SPA binding assay uses a reversible radioligand, which can bind to the desired pocket of the target protein. Although several FAAH ligands have been described, most of them are either degradable substrates or irreversible FAAH inhibitors, neither being very suitable for a binding assay.

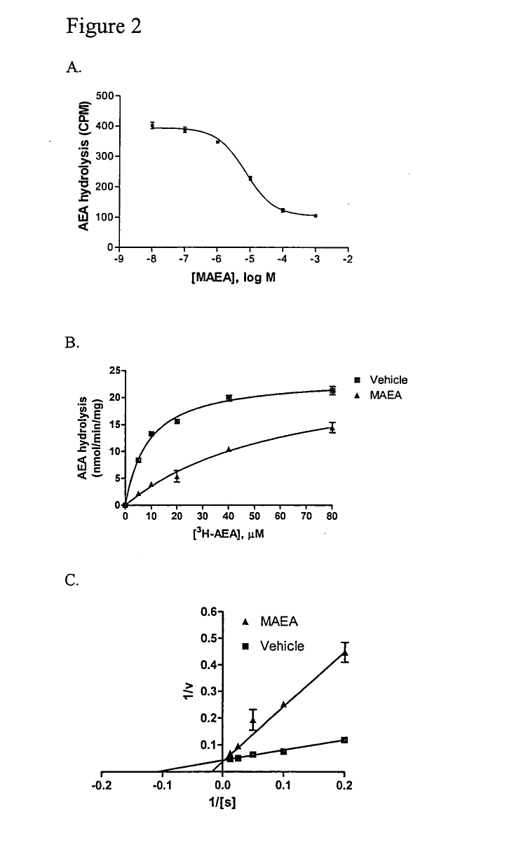

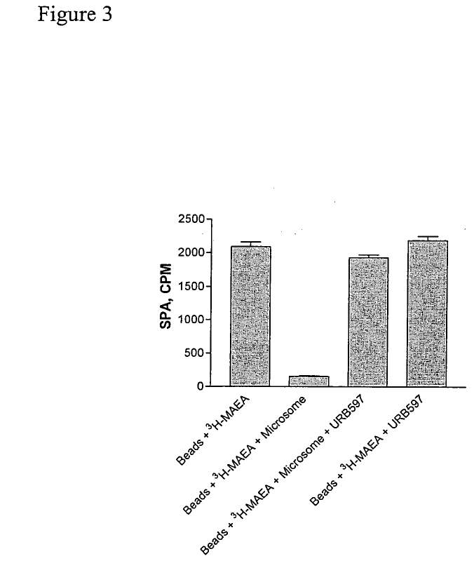

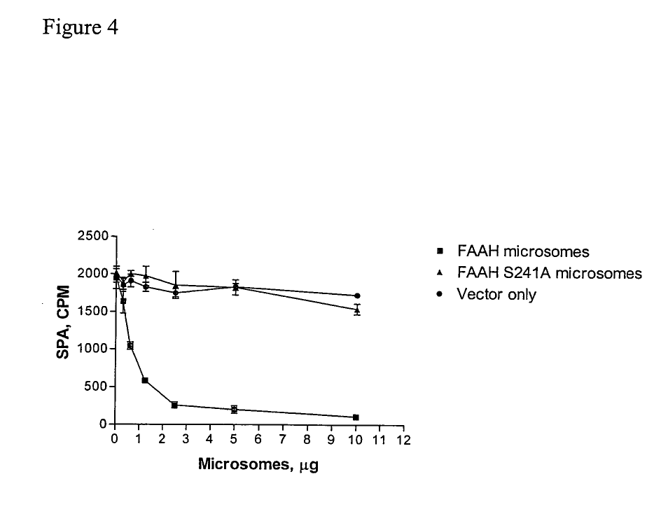

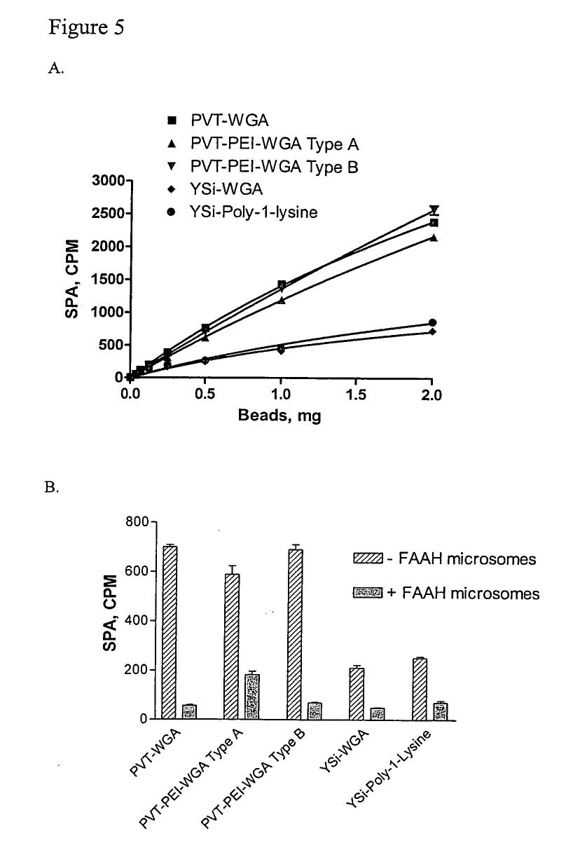

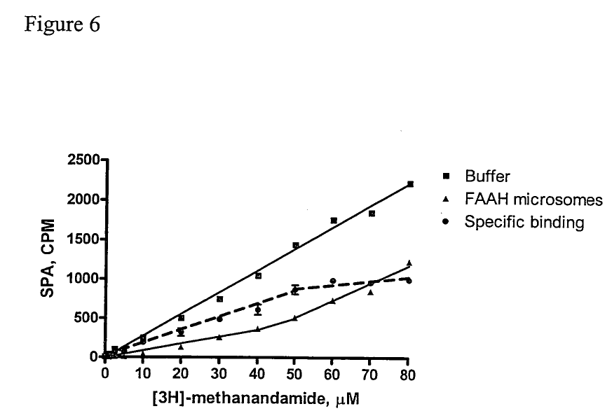

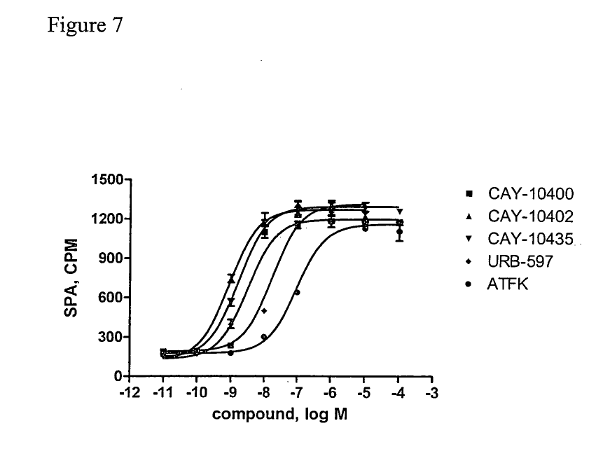

R(+)-methanandamide (R-(+)-arachidonyl-1-′-hydroxy-2′-propylamide, MAEA) is an anandamide analog with a methyl group at the 1′-position of the ethanolamido head group. It has been reported that MAEA has much greater resistance to enzymatic turnover and therefore has an increased metabolic stability in vivo (See Vasiliki Abadji, et al., (R)-Methanandamide: A Chiral Novel Anandamide Possessing Higher Potency and Metabolic Stability, J. Med. Chem., 1994, 37: 1889-1893; Khanolkar, A. D., et al., Head Group Analogs of Arachidonylethanolamide, the Endogenous Cannabinoid Ligand, J. Med. Chem., 1996, 39: 4515-4519 and Lang, W., et al., Substrate Specificity and Stereoselectivity of Rat Brain Microsomal Anandamide Amidohydrolase, J. Med. Chem., 1999, 42: 896-902). In hydrolysis experiments using FAAH microsomes, less than 1.5% of the [³H]-MAEA was degraded by 5 μg FAAH microsomes over 5 hours (data not shown). This relatively low turnover rate indicates that MAEA is resistant to FAAH hydrolysis. The effects of MAEA on FAAH-catalyzed anandamide hydrolysis was also examined using FAAH-containing microsomes. The results showed that MAEA inhibited FAAH in a dose dependent manner, with an IC₅₀ of 7.4 μM (FIG. 2a). The kinetic analysis of anandamide hydrolysis by FAAH-containing microsomes demonstrated that MAEA at 10 μM increased the apparent Kₘ for AEA from 9.1 μM to 62.3 μM, but no significant changes were observed for the Vₘₐₓ, (23.9 nmol/min/mg protein with vehicle compared to 25.82 nmol/min/mg protein in the presence of the compound) (FIG. 2b). The Lineweaver-Burk plot suggests that MAEA is a competitive FAAH inhibitor which interacts reversibly with the active site (substrate pocket) of the enzyme (FIG. 2c). Based on the kinetic analysis, [³H]-MAEA was selected as a radioligand to develop a SPA for FAAH.

### EXAMPLE 3

### FAAH SPA Binding Assay

Assays were performed by incubating 0.5 mg of PVT-WGA beads, 100 nM [3H]-MAEA, and 5 μg of FAAH-containing microsomes. Assays were incubated in the wells of 96-well plates. It was determined that these SPA assays required at least 2 hours to reach steady state (data not shown), thereafter assays were counted after 3 hours incubation.

As shown in FIG. 3, incubation of the WGA SPA beads with [³H]-MAEA produced SPA counts around ˜2100 cpm, which represented the non-specific binding of [³H]-MAEA to the SPA beads. When 5 μg of microsomes containing FAAH were included, the SPA counts decreased to ˜160 cpm, suggesting that ˜92% of total [³H]-MAEA was bound to FAAH microsomes and only ˜8% bound to WGA beads.

A selective FAAH inhibitor, URB-597, blocked the binding of [³H]-MAEA to the FAAH microsomes, as shown by the increase in SPA counts from 160 cpm to 1932 cpm. In contrast, the same concentration of URB-597 had no effect on the interaction of [³H]-MAEA and the WGA beads.

The nature of the interaction of the [³H]-MAEA and SPA bead was further explored in an experiment in which the assay components were diluted either 1 or 2 fold with the assay buffer before counting. Results showed that there was no obvious dilution-dependence of SPA signals in the experiment (data not shown). These results suggest that the SPA signals observed by the interaction of the radiolabel and the bead were not due to a non-proximity effect (NPE), which is caused by unbound isotopes coming close enough to stimulate the SPA bead when an excess of bead or radiolabel is present. NPEs are normally dilution-dependent. Without limiting the possible mechanisms, one presently preferred explanation is that the non-specific interaction between [³H]-MAEA and the WGA beads is due to the compound's lipophilic properties.

### EXAMPLE 4

### [³H]-MAEA Binds Specifically to the FAAH Microsomes

To further verify that [³H]-MAEA binds specifically to the FAAH microsomes, the binding activity of [³H]-MAEA to the microsomal fractions from CHO cells expressing either vector alone, FAAH, or inactive FAAH-S241A mutant was examined.

Western blot analysis using an antibody against FAAH showed similar expression levels of FAAH and FAAH-S241A proteins with no detectable level of endogenous FAAH in the vector transfected CHO cells (data not shown).

Titration curves with the three types of microsomes in the SPA assay are shown in FIG. 4. The SPA assay was carried out by mixing 0.5 mg of PVT-WGA beads, 100 nM [³H]-MAEA and different amounts of microsomes (derived from cells containing either vector only, FAAH, or FAAH-S241A mutant). Wild-type FAAH-containing microsomes, but not those containing vector or S241A mutant FAAH, captured the [³H]-MAEA in a dose-dependent manner as indicated by the decrease in SPA counts. The SPA counts are directly proportional to the concentration of free radiolabel. The binding reached saturation at 4-5 μg of microsomes containing FAAH per assay.

By comparison, microsomes from cells comprising vector only or inactive FAAH-S241A did not lead to any significant decrease in the SPA counts at concentrations up to 20 μg per well, suggesting no radioligand binding to these microsomes. The specific binding of [³H]-MAEA to the various microsomes at 5 μg were 90% for microsomes containing FAAH, 6.5% for those containing inactive FAAH-S241A, and 6.1% for microsomes prepared from cells containing only vector. These results indicate that the decrease in SPA counts is specific for the FAAH-containing microsomes.

### EXAMPLE 5

### Selection of SPA Beads in SPA Assay

A number of SPA beads have been developed by commercial suppliers. These beads differ, for example, in their composition and/or their surface molecules, which can be specific for the coupling of target proteins, membrane fractions, or cells. Comparisons of five types of SPA beads available from Amersham Bioscience was performed in the FAAH SPA assay as described above. Beads of types PVT-WGA, PVT-PEI-WGA type A, PVT-PEI-WGA type B, Ysi-WGA and Ysi-Poly-1-lysine beads were each used in comparative assays.

The results shown in FIG. 5a indicated that while all beads functioned, about 2-3 fold more [³H]-MAEA bound to the PVT beads (PVT-WGA, PVT-PEI-WGA type A, PVT-PEI-WGA type B) than to the Ysi beads (Ysi-WGA and Ysi-Poly-1-lysine). In the presence of FAAH-containing microsomes, PVT-WGA and PVT-PEI-WGA type B produced at least a 10-fold signal to noise ratio, whereas PVT-PEI-WGA type A, Ysi-WGA and YSI-Poly-1-lysine beads produced only a 2-fold window (FIG. 5b). These results suggest that while the assays can be performed in a variety of beads, presently PVT-WGA and PVT-PEI-WGA type B are preferred beads for use in FAAH SPA.

### EXAMPLE 6

### Binding of [³H]-MAEA to PVT-WGA SPA and FAAH-Containing Microsomes

The effect of the [³H]-MAEA concentration on the SPA signal was investigated by incubating increasing amounts of [³H]-MAEA with 0.5 mg of PVT-WGA beads (FIG. 6). The increase in SPA counts was linear with the concentration of [³H]-MAEA. No obvious saturation was observed up to 80 μM. This increase represented the total binding of [³H]-MAEA to the WGA beads.

Inclusion of 2 μg of microsomes comprising FAAH in the assay reduced the SPA counts accordingly (FIG. 6). The specific binding of [³H]-MAEA to the FAAH microsomes was calculated as the difference between the SPA counts with and without the FAAH containing microsomes. The specific binding reached saturation levels at the concentration of 50 μM of [³H]-MAEA. An estimated K_{d} of 25 μM is comparable to the IC₅₀ of 7.4 μM seen in the hydrolysis assay.

### EXAMPLE 7

### Effects of FAAH Inhibitors on the Binding of [³1H]-MAEA to FAAH-Containing Microsomes

To validate the method for screening for FAAH inhibitors, several well-known FAAH selective inhibitors including URB-597, CAY-10400, CAY-10402, and CAY-10435, as well as a nonselective inhibitor, ATFK, were tested in the FAAH SPA.

As shown in FIG. 7, all five compounds inhibited the binding of [³H]-MAEA to FAAH-containing microsomes in a concentration-dependent manner, at an IC₅₀ of 3.1 nM, 0.9 nM, 1.6 nM, 21.5 nM and 87 nM respectively. These values were compared with their activities in an anandamide hydrolysis assay. The data were found to correlate well (Table 1).

TABLE 1
Comparison of IC50 values of FAAH inhibitors
in the SPA and AEA hydrolysis assays

| | SPA | AEA hydrolysis |
|---|---|---|
| Compounds | (IC₅₀, nM) | (IC₅₀, nM) |
| CAY-10400 | 3.1 | 2.3 |
| CAY-10402 | 0.9 | 0.55 |
| CAY-10435 | 1.6 | 0.52 |
| URB-597 | 21.5 | 19.8 |
| ATFK | 87 | 10.1 |

The IC₅₀ values of compounds in the SPA were calculated from the data shown in FIG. 6. The AEA hydrolysis assays were performed according to the protocols described in Example 1. 0.5 μg of FAAH-containing microsomes per assay were incubated with 8 different concentrations (in a range from 1 μM to 10 μM) of compound in the presence of 1 μM [³H]-MAEA in 50 mM HEPES, pH 7.5, 1 mM EDTA, 0.1% BSA at room temperature for 2 hours. ³H-ethanolamine released from AEA hydrolysis was measured by liquid scintillation counting. Results were analyzed using a sigmoidal dose-response equation by GraphPad Prism 3.02. Numerical results are the mean of triplicate measurements from at least two independent experiments.

### EXAMPLE 8

### Effects of Various Assay Conditions on SPA Assay

The uniformity and signal to noise ratio of the microsome-based FAAH SPA assay was determined over multiple experiments in a 96-well format. The Z′ factor, which is a measure of the reproducibility of the assay and its compatibility for HTS, ranged from 0.73-0.8 suggesting that the assay is well-suited for high-throughput screening of candidate compounds.

The effect of DMSO on the FAAH SPA was analyzed by incubating 0.5 μg of FAAH-containing microsomes with 100 nM [³H]-MAEA and 0.5 mg of PVT-WGA beads, with DMSO concentrations that ranged from 0 to 10% (v/v). Results indicated that DMSO concentrations up to 10% of the final assay volume had no significant effect on the assay.

The effect of BSA in the SPA assay was also evaluated. BSA concentration of 0.1% of the assay volume was found to increase the reproducibility of the assay. Without limiting the successful effect to any one theory of operation, the presence of BSA might assist in maintaining the solubility of the highly lipophilic MAEA, minimize non-specific binding of the substrate to the walls of the assay wells, and/or improve the stability of the microsomes.

Each of the patent documents and scientific or technical publications disclosed hereinabove is incorporated by reference herein for all purposes.

Variations, modifications, and other implementations of what is described herein will occur to those of ordinary skill in the art without departing from the spirit and the essential characteristics of the present teachings. Accordingly, the scope of the invention is to be defined not by the preceding illustrative description but instead by the following claims, and all changes that come within the meaning and range of equivalency of the claims are intended to be embraced therein.

What is claimed:

1. A method of identifying a compound capable of binding to fatty acid amide hydrolase (FAAH), the method comprising:
incubating a membrane-associated FAAH with a labeled, known reversible inhibitor of FAAH activity in the presence of at least one type of scintillation proximity assay (SPA) bead and a test compound suspected of being capable of binding to the FAAH, wherein when the labeled known reversible inhibitor is bound to the FAAH, substantially no signal is generated from the SPA beads and wherein the presence of unbound labeled known reversible inhibitor in the assay medium causes the scintillation proximity beads to generate a detectable signal;
measuring a signal from the SPA beads;
comparing the signal so measured to a signal measured in a control assay incubated in the absence of the test compound; and
determining if the test compound binds to the FAAH by correlating an increase in signal with an increase in binding of the test compound.

2. The method of claim 1 wherein the membrane-associated FAAH comprises a lipid bilayer system.

3. The method of claim 2 wherein the lipid bilayer system is selected from a liposome, a microsome, a cell ghost, a cell membrane, or a cell membrane fraction.

4. The method of claim 3 wherein the lipid bilayer system is a microsome.

5. The method of claim 1 wherein the at least one type of SPA bead comprises at least one of yttrium silicate, polyvinyltoluene, wheat germ agglutinin, polylysine, and polyethyleneimine.

6. The method of claim 1 wherein at the at least one type of SPA bead comprises polyvinyltoluene and wheat germ agglutinin.

7. The method of claim 1 wherein the reversible inhibitor of FAAH is methanandamide, URB-597, CAY10400, CAY 10402, or CAY-10435.

8. The method of claim 7 wherein the reversible inhibitor is methanandamide.

9. The method of claim 1 wherein the reversible inhibitor is a substrate analog that is not substantially hydrolyzed by the FAAH enzyme.

10. The method of claim 1 wherein the labeled known reversible inhibitor is a radioactive labeled known reversible inhibitor comprising at least one of ¹⁴C and ³H.

11. The method of claim 1 wherein the detectable signal comprises an emission of light.

12. The method of claim 11 wherein the light comprises at least one visible wavelength.

13. The method of claim 1 wherein the test compound binds to the active site of the FAAH.

14. A kit for assaying a compound for its ability to bind to a FAAH enzyme, the kit comprising:
a membrane-associated FAAH;
at least one type of scintillation proximity assay (SPA) bead;
a labeled, known reversible inhibitor of FAAH; and
instructions for a scintillation proximity assay-based method for detecting a compound that binds to the FAAH enzyme.

15. The kit of claim 14 further comprising a reaction vessel and a detector for detecting a signal generated by the scintillation proximity beads, and optionally, a computer system for storing and analyzing results obtained from the assays.

16. The kit of claim 15 further comprising at least one known inhibitor as a standard.

17. The kit of claim 14 wherein the labeled inhibitor is [³H]—R(+)-methanandamide.

18. The kit of claim 17 wherein the at least one type of SPA bead comprises yttrium silicate, polyvinyltoluene, wheat germ agglutinin, polylysine, polyethyleneimine, or a combination thereof.

19. A kit for detecting a compound capable of binding to a FAAH enzyme, the kit comprising:
a vector comprising a FAAH gene for expressing in a cell;
at least one scintillation proximity assay bead;
a labeled, known competitive inhibitor of FAAH; and
instructions for a scintillation proximity assay-based method for detecting a compound that binds to the FAAH.

20. The kit of claim 19 wherein the vector comprises pHTOP-FAAH-His6 for expression in a mammalian cell.
